(19) United States
(12) Patent Application Publication
(10) Pub. No.: US 2007/0055370 A1
(43) Pub. Date: Mar. 8, 2007

(54) Packaging system for intraocular lens

(75) Inventors: Mark Sorochkin, Natzrat Ilit (IL); Moshe Barak, Adi (IL)

Correspondence Address: David Klein;DEKEL PATENT LTD, Beit HaRof'im, 18 Menuha VeNahala Street Room 27, Rehovot, IL

(73) Assignee: HANITA LENSES, Kibbutz Hanita (IL)

(21) Appl. No.: 10/572,055

(22) PCT Filed: Sep. 19, 2004
(86) PCT No.: PCT/IL04/00860

§ 371 Date: Mar. 15, 2006

(30) Foreign Application Priority Data
Sep. 17, 2003 (IL) 157979

(51) Int. Cl. A61F 2/16

(52) U.S. Cl. 623/6.12

(57) ABSTRACT

A packaging system for an IOL including an IOL pre-packaged in a liquid medium in a load chamber of an IOL delivery device, the IOL delivery device including an injection port through which the IOL is expellable.

### FIELD OF THE INVENTION

This invention relates generally to intraocular lenses and injectors and, more particularly, to packaging systems and methods for holding intraocular lenses until eventual injection into an eye.

### BACKGROUND OF THE INVENTION

As is well known, intraocular lenses (IOLs) may be used to replace the natural lens of the eye when the natural lens becomes damaged or otherwise incapable of functioning. A typical IOL includes an optic or lens body for focusing light toward the retina of the eye, and one or more fixation members or haptics for fixing the IOL in the desired position within the eye.

The IOL may be implanted directly into the eye through a small incision formed in the ocular tissue of the eye. To fit through this small incision, optics are generally designed to be deformed, e.g., folded, rolled or the like, to a relatively small profile and then allowed to substantially return to their original shape within the eye.

The IOL is typically provided to the surgeon or other medical personnel in a packaging system that contains the IOL. These packaging devices protect the IOL during handling and transportation to the surgical site and may even allow for accurately folding the IOL along the optic diameter in order to reduce its size. Some of these systems specifically fold the IOL in preparation for grasping by the forceps or other surgical instrument.

A very useful and common technique for inserting an IOL into the eye includes the use of an IOL injector usually with a cartridge. These IOL injectors include a load chamber which is connected to an injection port. The load chamber includes an openable first lumen for receiving the IOL. Closure of this first lumen folds the IOL and maintains the IOL in a folded state. The injection port includes a small diameter distal tip which is insertable into the incision within the eye. The IOL is transferable from the load chamber through the injection port and into the eye. These IOL injectors simplify the placement of the IOL within the eye and reduce chances of surgeon error.

However, various problems may arise while removing the IOL from its sterile storage container, manually folding the IOL, manually placing the folded IOL into the insertion device, and expelling the IOL into the eye. The professional must not drop the IOL, or damage the IOL by improperly folding it. Next, the professional must properly insert the folded IOL into the delivery device without damaging it. The IOL must be positioned in the insertion device in the proper orientation so that the IOL and associated haptics are not damaged during the process of expelling the IOL into the eye. Increased handling increases the risk of damaging and/or contaminating the IOL and/or injector. At all times, the sterility of the IOL must be maintained. Once the sterility of the IOL is broken, it must be discarded. Additionally, the folding and inserting process can be time consuming.

U.S. Pat. No. 6,447,519 to Brady et al. attempts to solve this problem by providing a package system (that is, holding apparatus) for an intraocular lens injector. The holding apparatus includes a load or loading chamber, and a pair of generally opposing wings, wherein the loading chamber is maintained in an open position. The holding apparatus comprises a housing sized and adapted to receive the intraocular lens injector. Holding elements are secured to the housing that stabilize the position of the intraocular lens injector relative to the housing, with the loading chamber in the open position. However, the IOL must still be manually manipulated during placing in the loading chamber.

U.S. Pat. No. 6,228,094 to Erdman also attempts to solve the problem by providing a container that stores an IOL, and which also functions as an “automatic” IOL folding and inserting device. The sterile IOL is positioned within the container. The container also includes a lens folding mechanism. The container may be coupled with a lens delivery device. Thus, the lens container serves the dual purpose of storing the IOL, and folding and inserting the IOL into the lens delivery device.

### SUMMARY OF THE INVENTION

The present invention seeks to provide novel packaging systems and methods for holding intraocular lenses (IOLs) until eventual injection into an eye, as described in detail hereinbelow. The IOL may be held in a liquid medium (e.g., aqueous solution) at all times until injection in the eye, which may be particularly useful for a hydrophilic IOL.

There is thus provided in accordance with an embodiment of the present invention a packaging system for an IOL including an IOL pre-packaged in a liquid medium in a load chamber of an IOL delivery device, the IOL delivery device including an injection port through which the IOL is expellable.

In accordance with an embodiment of the present invention an injector expeller assembly is provided which may be connected to an interface port of the IOL delivery device, the injector expeller assembly adapted to expel the IOL from the load chamber to exit through the injection port. A carrying case may be provided in which the IOL delivery device, with the IOL pre-packaged therein, and the injector expeller assembly are disposed within.

In accordance with an embodiment of the present invention the IOL delivery device further includes a deformer arranged to move in the IOL delivery device with respect to the load chamber and adapted to deform the IOL.

In accordance with an embodiment of the present invention movement of the deformer towards the load chamber pushes the IOL against an inner peripheral wall of the load chamber, and further movement of the deformer towards the load chamber curls the IOL about the inner peripheral wall of the load chamber.

In accordance with another embodiment of the present invention movement of the deformer towards the load chamber squeezes and folds the IOL. Further in accordance with an embodiment of the present invention the deformer may include a pair of push arms arranged to move symmetrically inwards towards the load chamber and squeeze and fold the IOL.

There is also provided in accordance with an embodiment of the present invention a method of preparing an IOL for insertion into an eye, the method including providing an IOL pre-packaged in a load chamber of an IOL delivery device, the IOL delivery device including an injection port through which the IOL is injectable into an eye.

### BRIEF DESCRIPTION OF THE DRAWINGS

The present invention will be understood and appreciated more fully from the following detailed description taken in conjunction with the appended drawings in which:

FIGS. 1, 2 and 3 are simplified pictorial, side sectional and end sectional illustrations, respectively, of a packaging system for an intraocular lens (IOL), in accordance with an embodiment of the present invention;

FIG. 4 is a simplified illustration showing an IOL pre-packaged in the packaging system of FIG. 1, in accordance with an embodiment of the present invention;

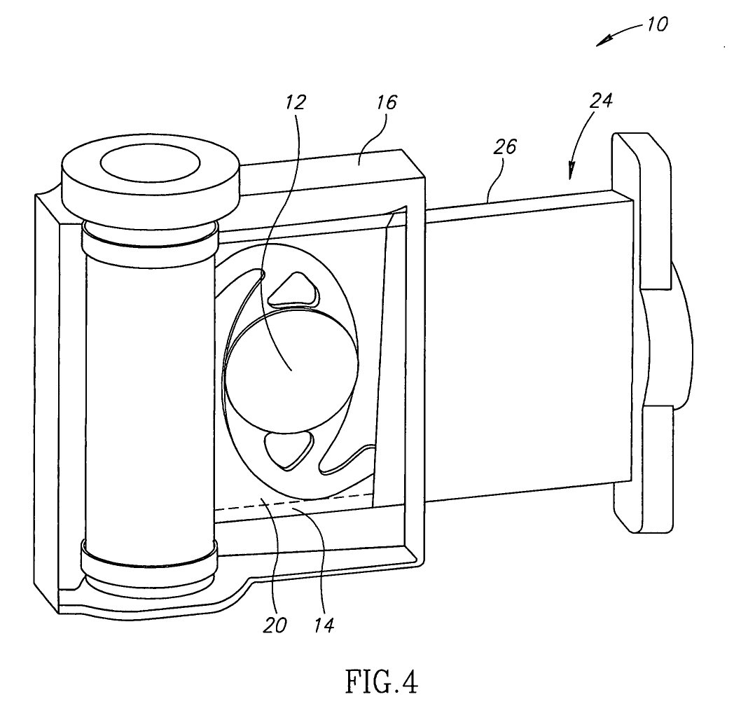

FIG. 5 is a simplified illustration of the pre-packaged IOL packaging system and an injector expeller assembly provided together in a carrying case, in accordance with an embodiment of the present invention;

FIGS. 6 and 7 are simplified illustrations of the pre-packaged IOL packaging system attached to the injector expeller assembly, respectively before and after deforming the IOL;

FIG. 8 is a simplified illustration of pushing a plunger of the injector expeller assembly to expel the IOL from the chamber of the packaging system;

FIGS. 9 and 10 are simplified pictorial illustrations of a packaging system for an intraocular lens (IOL), in accordance with another embodiment of the present invention, respectively before and after deforming the IOL;

FIG. 11 is a simplified sectional illustration of the pre-packaged IOL packaging system of FIGS. 9 and 10 attached to the injector expeller assembly, in accordance with an embodiment of the present invention; and

FIG. 12 is a simplified pictorial illustration of the packaging system of FIGS. 9 and 10, is which a guide member is provided with a locking device that locks a push arm, in accordance with an embodiment of the present invention.

### DETAILED DESCRIPTION OF PREFERRED EMBODIMENTS

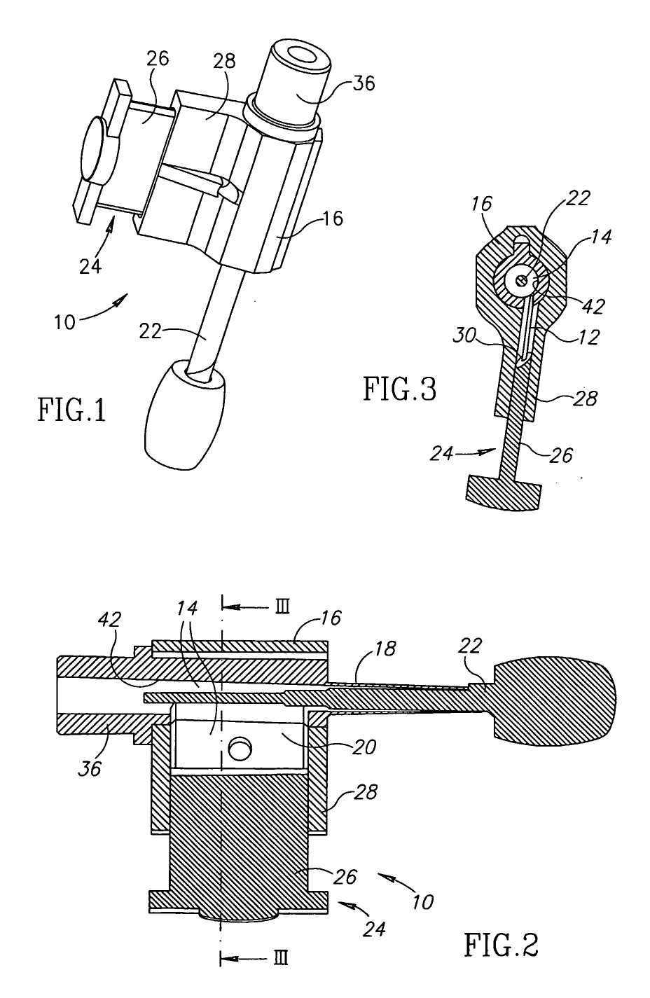

Reference is now made to FIGS. 1-3, which illustrate a packaging system 10 for an IOL 12, constructed and operative in accordance with an embodiment of the present invention.

IOL 12 may be made of a relatively soft and flexible biologically inert optical material, such as but not limited to, silicone, acrylic, collagen, hydrogel, hyaluronic acid (including the sodium, potassium and other salts thereof), and polysulfones. IOL 12 may be pre-packaged in a load chamber 14 of an IOL delivery device 16. IOL delivery device 16 may be constructed of any suitable material, such as but not limited to, polycarbonate or polypropylene.

IOL delivery device 16 may comprise an injection port 18 through which IOL 12 may be expelled and injected into an eye. IOL 12 may be pre-packaged in a liquid medium 20 (such as but not limited to, an aqueous saline solution) in load chamber 14. Plugs (not shown) may be provided with the pre-packaged packaging system 10 that seal liquid medium 20 in load chamber 14. A rod 22, constructed of stainless steel or a plastic, may initially extend into load chamber 14. The entire packaging system 10, including IOL 12 and IOL delivery device 16, may be sterilized as one unit. This may reduce the potential risk of one item failing sterilization while another item passed sterilization, which eventually may lead to a health risk to the patient.

A deformer 24 may be arranged to move in IOL delivery device 16 with respect to load chamber 14. Deformer 24 is used to deform IOL 12 prior to insertion into an eye, as is described further hereinbelow. In the illustrated embodiment, deformer 24 comprises a pushing element 26 that slides in an extension 28 of IOL delivery device 16. An end 30 of pushing element 26 (FIG. 3) may be chamfered to accommodate pushing IOL 12.

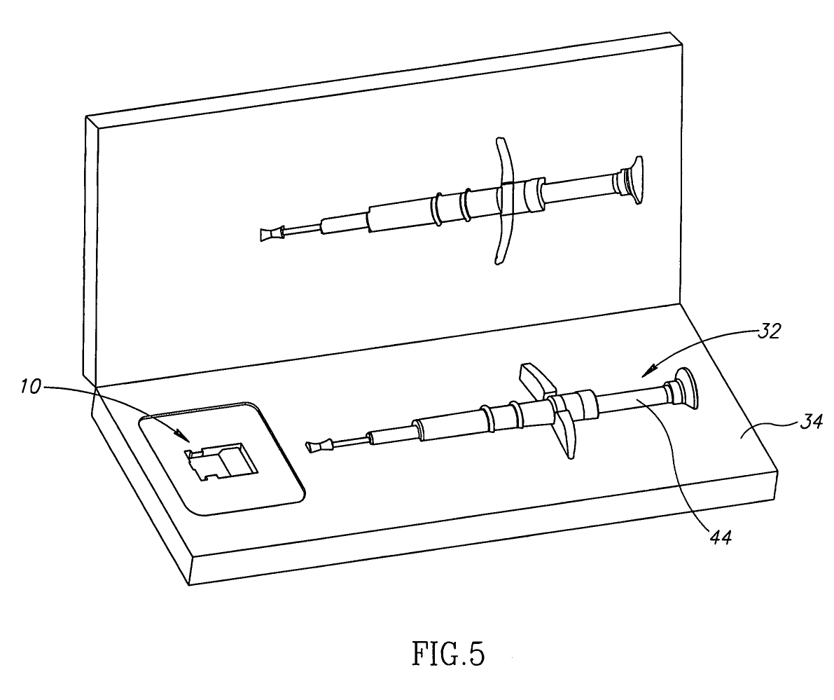

Reference is now made to FIG. 5. An injector expeller assembly 32 may be provided for expelling IOL 12 from load chamber 14. Injector expeller assembly 32 may be packaged together with the packaging system 10 (including IOL delivery device 16 with IOL 12 pre-packaged therein) in a carrying case 34. In this manner, packaging system 10 may have IOL 12 pre-packaged in liquid medium 20, while injector expeller assembly 32 is kept dry. Injector expeller assembly 32 may be connected to an interface port 36 of IOL delivery device 16.

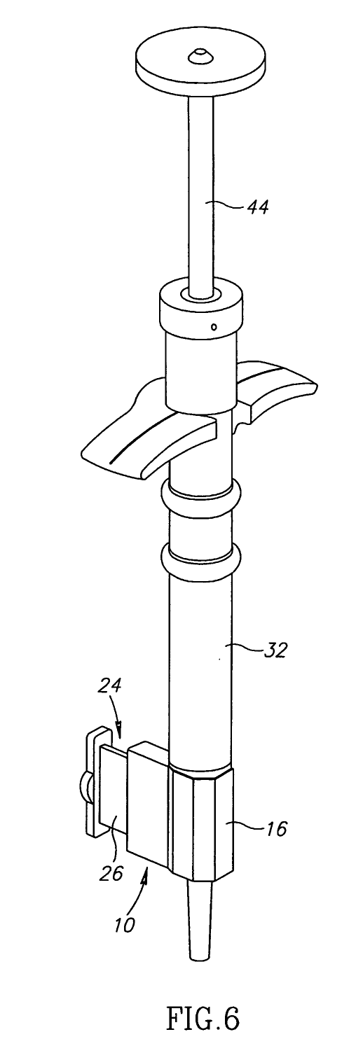

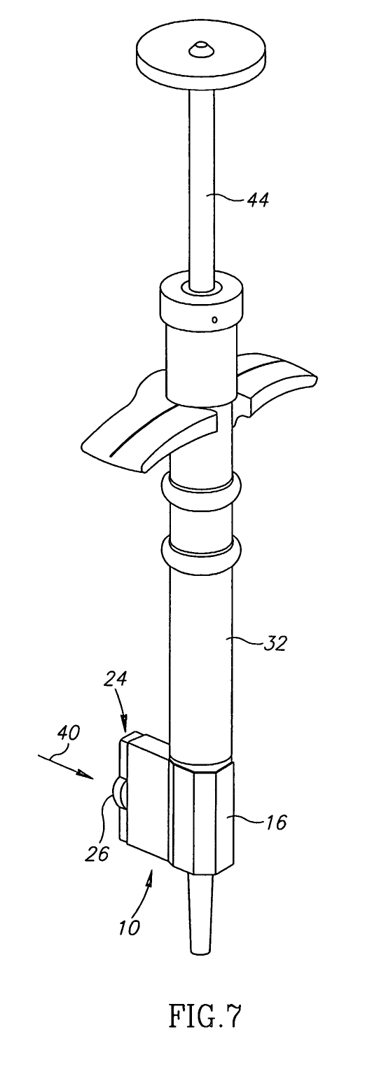

Reference is now made to FIG. 6, which illustrates packaging system 10 attached to a distal end of injector expeller assembly 32. Deformer 24 has not yet been actuated to deform IOL 12. Referring to FIG. 7, pushing element 26 may be pushed into extension 28 of IOL delivery device 16, as indicated by an arrow 40. Referring to FIGS. 2 and 3, the initial movement of pushing element 26 in the direction of arrow 40 towards load chamber 14 pushes IOL 12 against an inner peripheral wall 42 of load chamber 14. Further movement of pushing element 26 in the direction of arrow 40 towards load chamber 14 curls IOL 12 about the inner peripheral wall 42 of load chamber 14. Thus IOL 12 is rolled (or otherwise folded or deformed) into a compact orientation ready for insertion into an eye (not shown). In FIG. 8, a plunger 44 of injector expeller assembly 32 may now be pushed distally in the direction of an arrow 46 to expel IOL 12 from load chamber 14 into an eye (not shown).

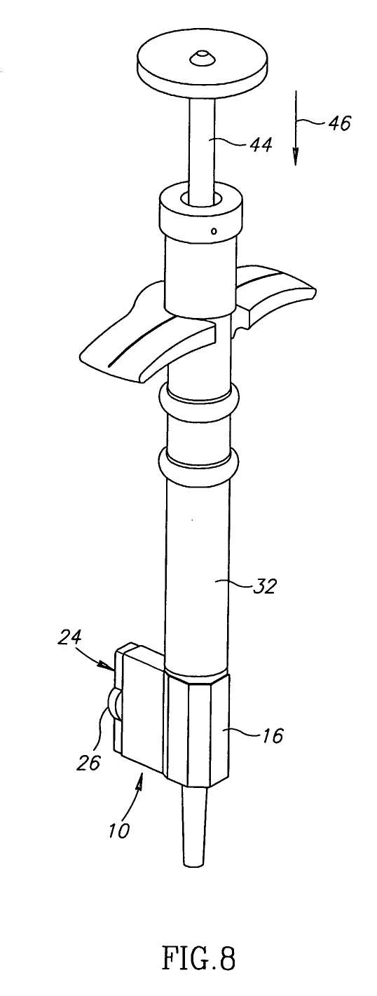

Reference is now made to FIGS. 9 and 10, which illustrate a packaging system 50 for an IOL 12, constructed and operative in accordance with another embodiment of the present invention.

IOL 12 may be pre-packaged in a load chamber 54 of an IOL delivery device 56. IOL 12 may be disposed in a liner 52 in load chamber 54. Liner 52 may be constructed of any suitable material, such as but not limited to, a medically safe elastomer or soft plastic. IOL delivery device 56 may be constructed of any suitable material, such as but not limited to, polycarbonate or polypropylene.

IOL delivery device 56 may comprise an injection port 58 through which IOL 12 may be expelled and injected into an eye. IOL 12 may be pre-packaged in liquid medium 20 in load chamber 54. Plugs (not shown) may be provided with the pre-packaged packaging system 50 that seal liquid medium 20 in load chamber 54.

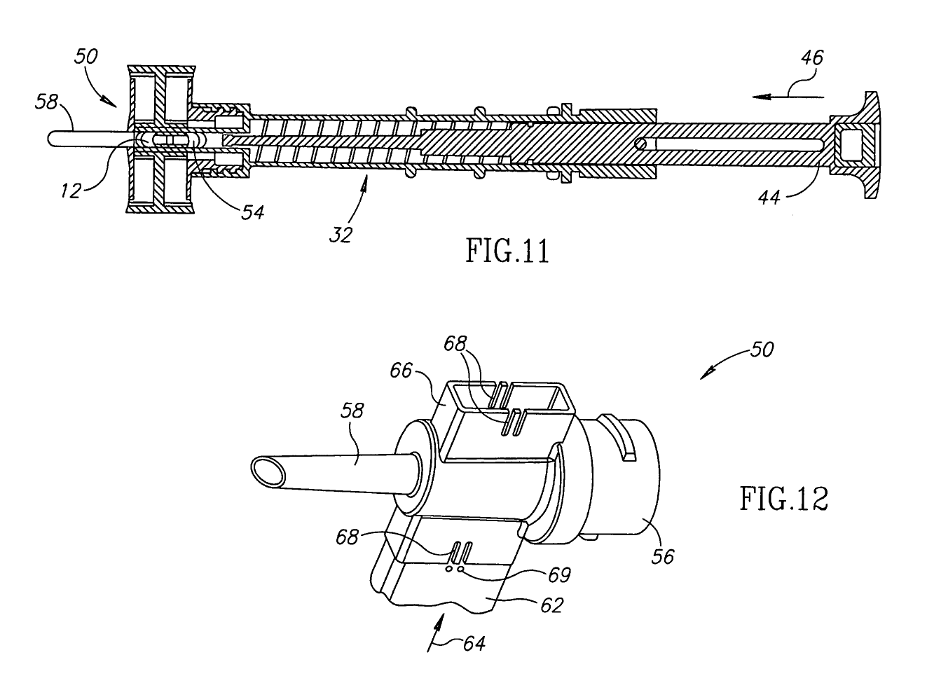

A deformer 60 may be arranged to move in IOL delivery device 56 with respect to load chamber 54 to deform IOL 12 prior to insertion into an eye. In the illustrated embodiment, deformer 60 comprises a pair of push arms 62 arranged to move symmetrically inwards towards load chamber 54 and squeeze and fold IOL 12, as indicated by arrows 64 in FIG. 10. FIG. 10 illustrates one embodiment in which the push arms 62 slide simply in guide members 66 that protrude sideways from IOL delivery device 56. FIG. 12 shows an alternative embodiment, in which guide member 66 is provided with a locking device 68, such as but not limited to, locking channels formed in guide member 66. Push arms 62 (partially shown in FIG. 12) may be provided with complementary shaped lugs 69 which lockingly mate with locking device 68. In this manner, locking device 68 locks push arms 62 in place when push arms 62 are moved sufficiently inwards towards load chamber 54. Thus, a user can push the push arms 62 to the locked position and not have to continuously squeeze them. It is appreciated that this is just one example of a locking arrangement and the invention is not limited to the illustrated arrangement.

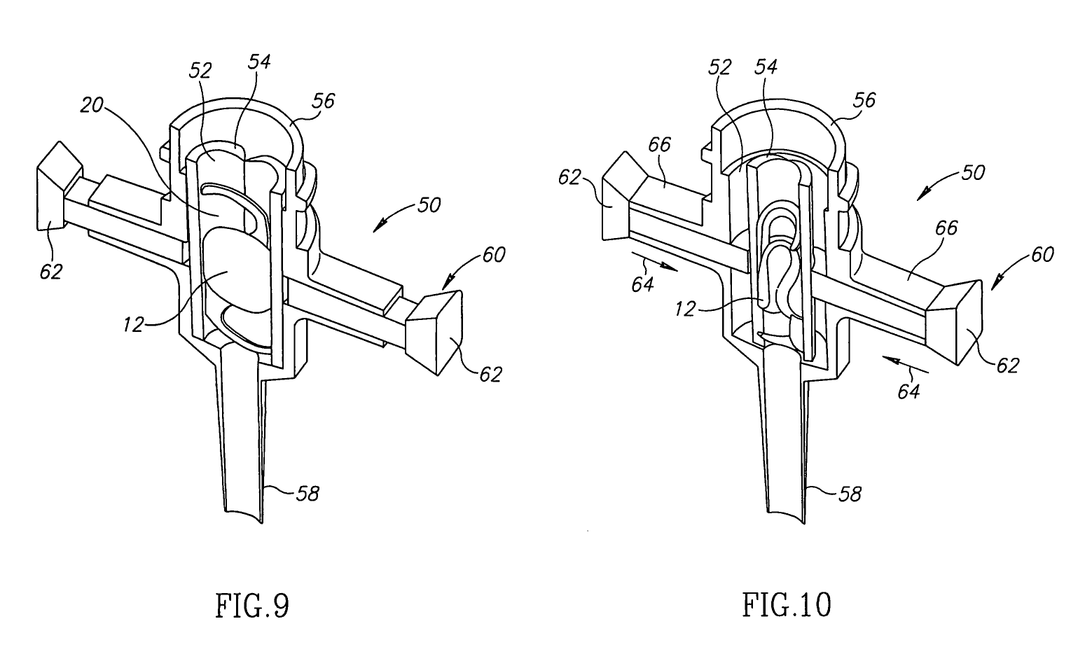

Reference is now made to FIG. 11, which illustrates the packaging system 50 of FIGS. 9 and 10 attached to the distal end of injector expeller assembly 32, in accordance with an embodiment of the present invention. As similarly described hereinabove with reference to FIG. 8, after deforming IOL 12, plunger 44 of injector expeller assembly 32 may be pushed distally in the direction of arrow 46 to expel IOL 12 from load chamber 54 into an eye (not shown).

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A packaging system for an intraocular lens (IOL) comprising:
an IOL pre-packaged in a liquid medium in a load chamber of an IOL delivery device, said IOL delivery device comprising an injection port through which said IOL is expellable.

2. The packaging system according to claim 1, wherein said IOL and said IOL delivery device are sterilized as one unit.

3. The packaging system according to claim 1, further comprising an injector expeller assembly connectable to an interface port of said IOL delivery device, said injector expeller assembly adapted to expel said IOL from said load chamber to exit through said injection port.

4. The packaging system according to claim 3, further comprising a carrying case in which said IOL delivery device, with said IOL pre-packaged therein, and said injector expeller assembly are disposed within.

5. The packaging system according to claim 1, further comprising a rod extending into said load chamber.

6. The packaging system according to claim 1, wherein said IOL delivery device further comprises a deformer arranged to move in said IOL delivery device with respect to said load chamber and adapted to deform said IOL.

7. The packaging system according to claim 6, wherein movement of said deformer towards said load chamber pushes said IOL against an inner peripheral wall of said load chamber, and further movement of said deformer towards said load chamber curls said IOL about the inner peripheral wall of said load chamber.

8. The packaging system according to claim 6, wherein movement of said deformer towards said load chamber squeezes and folds said IOL.

9. The packaging system according to claim 8, wherein said deformer comprises a pair of push arms arranged to move inwards towards said load chamber and squeeze and fold said IOL.

10. The packaging system according to claim 9, wherein said push arms slide in guide members that protrude sideways from said IOL delivery device, said guide members being provided with a locking device adapted to lock said push arms in place when said push arms are moved sufficiently inwards towards said load chamber.

11. A method of preparing an IOL for insertion into an eye, the method comprising:
providing an IOL pre-packaged in a liquid medium in a load chamber of an IOL delivery device, said IOL delivery device comprising an injection port through which said IOL is injectable into an eye.

12. The method according to claim 11, further comprising sterilizing said IOL and said IOL delivery device as one unit.

13. The method according to claim 11, further comprising connecting an injector expeller assembly to an interface port of said IOL delivery device, said injector expeller assembly adapted to expel said IOL from said load chamber to exit through said injection port.

14. The method according to claim 13, further comprising deforming said IOL prior to expelling said IOL from said load chamber.

15. The method according to claim 14, wherein said deforming comprises pushing and curling said IOL against an inner peripheral wall of said load chamber.

16. The method according to claim 14, wherein said deforming comprises squeezing and folding said IOL.
